(19) United States
(12) Patent Application Publication
(10) Pub. No.: US 2022/0128553 A1
(43) Pub. Date: Apr. 28, 2022

(54) IMMUNOCHROMATOGRAPHY ANALYSIS DEVICE FOR DETECTING DENGUE VIRUS

(71) Applicant: TANAKA KIKINZOKU KOGYO K.K., Tokyo (JP)

(72) Inventors: Keita SUZUKI, Kanagawa (JP); Hisahiko IWAMOTO, Kanagawa (JP)

(73) Assignee: TANAKA KIKINZOKU KOGYO K.K., Tokyo (JP)

(21) Appl. No.: 17/427,569

(22) PCT Filed: Jan. 31, 2020
(86) PCT No.: PCT/JP2020/003813

§ 371 Date: Jul. 30, 2021

(30) Foreign Application Priority Data
Jan. 31, 2019 (JP) 2019-016494

(51) Int. Cl. G01N 33/543; G01N 33/569

(52) U.S. Cl. CPC G01N 33/54388; G01N 2333/185; G01N 33/56983

(57) ABSTRACT

The invention relates to an immunochromatography analysis device which enables simple and rapid diagnosis of dengue virus infection, and an object thereof is to provide an immunochromatography analysis device which can reduce a cross-reaction with a virus belonging to Flaviviridae other than dengue virus and which can specifically detect dengue virus. The invention relates to an immunochromatography analysis device for detecting dengue virus including a sample application part, a labeling substance-holding part, a chromatography medium part having a detection part and an absorption part, wherein the labeling substance-holding part contains a first antibody which recognizes the amino acid sequence of SEQ ID NO: 2 that is present in the whole amino acid sequence of dengue virus NS1 of SEQ ID NO: 1, and the detection part contains a second antibody which recognizes the three-dimensional structure of dengue virus NS1.

### CROSS-REFERENCE TO RELATED PATENT APPLICATIONS

This application is a National Phase Entry of International Patent Application No. PCT/JP2020/003813, filed Jan. 31, 2020, which claims priority of Japanese Patent Application No. 2019-016494, filed Jan. 31, 2019, both of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 30, 2021, is named T53363790KF4_SEQUENCE_LISTING.txt and is 14,458 bytes in size.

### TECHNICAL FIELD

The present invention relates to an immunochromatography analysis device for detecting dengue virus, an immunochromatography analysis kit and an immunochromatography analysis method.

### BACKGROUND

Dengue virus, which causes dengue virus infection, is a virus belonging to Flaviviridae (Flavi virus) together with Zika virus, Japanese encephalitis virus, West Nile virus and the like.

Dengue virus is transmitted by mosquitos and causes dengue virus infection such as dengue fever and dengue hemorrhagic fever. In the beginning, dengue virus infection had been seen as a problem in the tropical zone and the subtropical zone. Cases of dengue fever have been reported recently also in Japan, and dengue virus has been detected also from the native mosquitos (NPL 1).

Effective medications or vaccines against dengue virus infection have not been developed so far. To prevent the spread of dengue virus infection, diagnosis of dengue virus infection at an early stage is desired.

Diagnosis of dengue virus infection has been made so far by detecting the virus gene in the blood by RT-PCR or by detecting a nonstructural protein antigen (NS1 antigen) with an immunochromatographic reagent through a blood test, a urine test or a saliva test. As a serological method, an antibody (IgM, IgG or the like) to dengue virus that a person with dengue virus infection has in the body is detected by ELISA method or a fluorescent antibody technique.

### SUMMARY OF THE INVENTION

### Technical Problem

The diagnosis of dengue virus infection by RT-PCR, however, requires special equipment, reagents and the like and thus is expensive. Also, the inspection requires time of half a day to around a day, and it takes a long time to obtain the inspection results.

In the method of detecting an antibody to dengue virus, diagnosis of dengue virus infection is made indirectly by detecting IgM or IgG that is produced in the human body after a certain lapse of time following the infection with dengue virus, rather than directly detecting dengue virus. Thus, the method is not suitable for diagnosing dengue virus infection at an early stage. Moreover, because the method is not for directly detecting dengue virus, it is difficult during diagnosis to distinguish from infections with, for example, Zika virus, Japanese encephalitis virus and West Nile virus, which belong to Flaviviridae like dengue virus. Accordingly, means for diagnosing dengue virus infection from an early stage of infection by directly detecting dengue virus is desired.

The practical use of the detection of an NS1 antigen with an immunochromatographic reagent is difficult because of the cross-reactivity with the viruses belonging to Flaviviridae like dengue virus and because of the low sensitivity in the case of a reagent with low cross-reactivity. Therefore, means which is not cross-reactive to viruses with similar endemic areas and similar symptoms and which can rapidly detect dengue virus with a high sensitivity is desired.

Thus, an object of the invention is to provide an immunochromatography analysis device which enables simple and rapid diagnosis of dengue virus infection. Moreover, another object is to provide an immunochromatography analysis device which can find dengue virus infection at an early stage by directly detecting dengue virus itself and which can reduce a cross-reaction with a virus belonging to Flaviviridae other than dengue virus and specifically detect dengue virus.

### Solution to Problem

As a result of intensive study to solve the problems, the present inventors have found that the problems can be solved using antibodies that recognize dengue virus nonstructural protein NS1 (also simply called dengue virus NS1 or NS1 below) in an immunochromatography analysis device, and the inventors have thus completed the invention.

That is, the invention is as follows.
1. An immunochromatography analysis device for detecting dengue virus in an analyte, including a sample application part, a labeling substance-holding part, a chromatography medium part having a detection part and an absorption part, wherein the labeling substance-holding part contains a first antibody which recognizes the amino acid sequence of SEQ ID NO: 2 that is present in the whole amino acid sequence of dengue virus NS1 of SEQ ID NO: 1, and the detection part contains a second antibody which recognizes the three-dimensional structure of dengue virus NS1.
2. The immunochromatography analysis device according to 1 above, wherein the detection part further contains an antibody which recognizes the amino acid sequence of SEQ ID NO: 2 that is present in the whole amino acid sequence of dengue virus NS1 of SEQ ID NO: 1.
3. An immunochromatography analysis kit including the immunochromatography analysis device according to 1 or 2 above and an analyte dilution solution for diluting and developing the analyte.
4. An immunochromatography analysis method for detecting dengue virus in an analyte using the immunochromatography analysis kit according to 3 above, wherein the immunochromatography analysis method includes the following steps (1) to (4), (1) a step of applying an analyte-containing solution obtained by diluting the analyte with the analyte dilution solution as a sample to the sample application part, (2) a step of causing the first antibody which recognizes the amino acid sequence of SEQ ID NO: 2 that is present in the whole amino acid sequence of dengue virus NS1 of SEQ ID NO: 1 and which is held in the labeling substance-holding part to recognize dengue virus in the analyte, (3) a step of developing the analyte and the first antibody as a mobile phase on the chromatography medium part, and (4) a step of detecting the dengue virus in the developed mobile phase with the second antibody which recognizes the three-dimensional structure of dengue virus NS1 contained in the detection part.

### Advantageous Effects of Invention

The immunochromatography analysis device of the invention has a labeling substance-holding part and a detection part. The labeling substance-holding part contains a first antibody which recognizes the amino acid sequence of SEQ ID NO: 2 that is present in the whole amino acid sequence of a nonstructural protein, NS1, which dengue virus has, and the detection part contains a second antibody which recognizes the three-dimensional structure of dengue virus NS1. As a result, a cross-reaction with a virus or the like other than dengue virus can be inhibited, and dengue virus can be detected rapidly and specifically.

### BRIEF DESCRIPTION OF THE DRAWINGS

FIG. 1 is a cross section for explaining the structure of the immunochromatography analysis device of an embodiment of the invention.

FIG. 2 shows the results of a hydrophilicity plot. The vertical axis shows the score for hydrophilicity, and the horizontal axis shows the amino acid position in the amino acid sequence of SEQ ID NO: 1 from the N-terminus.

FIG. 3 shows the results of a competitive inhibition ELISA test. The vertical axis shows the absorbance.

FIG. 4 shows the results of a dot blotting.

FIG. 5 shows the results of western blotting.

FIG. 6 shows the results of measurement of color intensities of the detection parts after dropping and developing analyte-containing solutions on the sample application parts of immunochromatography analysis devices.

FIG. 7 shows the results of evaluation of cross-reactivities using immunochromatography analysis kits.

FIG. 8 shows the results of S/N ratios determined by a half-strip assay.

### DETAILED DESCRIPTION

Embodiments for carrying out the invention are explained below.

In this description, “immobilize” means that an antibody is arranged on a carrier such as a membrane in a manner that the antibody does not move, and “hold” means that an object is arranged in a manner that the object can move in a carrier such as a membrane or on the surface thereof.

In this description, that an antibody “recognizes” a specific protein means that the antibody binds to a part of the amino acid sequence that the protein has through the antigen-antibody reaction. Moreover, that an antibody “recognizes” a specific amino acid sequence means that the antibody binds to the whole or a part of the specific amino acid sequence through the antigen-antibody reaction.

Whether or not an antibody binds to the whole or a part of a specific amino acid sequence through the antigen-antibody reaction can be determined by a known immunoassay. When immunoassays are classified by the method of immunoassay, examples thereof include sandwich assay, competitive assay, agglutination assay and the like. When immunoassays are classified by the label used, examples thereof include fluorescence assay, enzyme assay, radiation assay and the like. The antigen-antibody reaction can be identified by any of the immunoassays.

The analyte that can be used for the invention is not particularly limited as long as the analyte may contain dengue virus. Examples of the analyte include serum, plasma, whole blood, semen, spinal fluid and the like of an individual infected with dengue virus. The analyte is preferably whole blood, serum or plasma in view of the rapid diagnosis.

<Immunochromatography Analysis Device>

The immunochromatography analysis device of the invention has a sample application part to which a sample containing an analyte (also simply called a sample below) is applied, a labeling substance-holding part holding a labeling substance, a chromatography medium part having a detection part for detecting dengue virus and an absorption part for absorbing a liquid which has passed through the detection part.

The immunochromatography analysis device of the invention is characterized in that the labeling substance-holding part contains a first antibody which recognizes the amino acid sequence of SEQ ID NO: 2 that is present in the whole amino acid sequence of a nonstructural protein, NS1, of dengue virus of SEQ ID NO: 1 and that the detection part contains a second antibody which recognizes the three-dimensional structure of dengue virus NS1.

Dengue virus is classified into four virus types based on the serotypes, namely DENV-1, DENV-2, DENV-3 and DENV-4, and it is suggested that the protein involves in the virus replication and the like as in other flaviviruses. NS1 of DENV-1 is composed of the amino acid sequence of total 352 amino acid residues shown in SEQ ID NO: 1 and has high homology to the NS of the other serotypes.

Regarding the amino acid sequences of the NS of the other serotypes, the amino acid sequence of NS1 of DENV-2, the amino acid sequence of NS1 of DENV-3 and the amino acid sequence of NS1 of DENV-4 are shown in SEQ ID NO: 9, SEQ ID NO: 10 and SEQ ID NO: 11, respectively.

In the whole amino acid sequence of NS1 of SEQ ID NO: 1, the amino acid sequence of SEQ ID NO: 3 is the 12nd to 35th amino acid residues, and the amino acid sequence of SEQ ID NO: 2 is the 100th to 121st amino acid residues. The amino acid sequence of SEQ ID NO: 4 is the 135th to 151st amino acid residues, and the amino acid sequence of SEQ ID NO: 5 is the 193rd to 222nd amino acid residues. The amino acid sequence of SEQ ID NO: 6 is the 248th to 271st amino acid residues, and the amino acid sequence of SEQ ID NO: 7 is the 282nd to 306th amino acid residues. The amino acid sequence of SEQ ID NO: 8 is the 325th to 352nd amino acid residues.

The NS1 which the first antibody and the second antibody recognize may be a natural protein isolated from the virus or a recombinant protein produced based on a known nucleotide sequence of a gene encoding NS1. Moreover, the NS1 which the first antibody and the second antibody recognize may be one which has been isolated/purified from the components constituting the virus or unpurified one. When the NS1 is not isolated, the NS1 may be NS1 derived from the virus which has been treated with a surfactant so that the NS1 and the antibodies come into contact easily.

In the invention, using antibodies recognizing especially NS1 of various proteins that dengue virus has because an amino acid mutation does not relatively easily occur and because the specificity and the detection sensitivity are improved, a cross-reaction with a virus or the like other than dengue virus can be inhibited, and dengue virus can be detected specifically. Moreover, because NS1 is a protein which stays longer in the blood than the virus gene, NS1 can be detected for a longer period after the development of a symptom.

The labeling substance-holding part in the immunochromatography analysis device of the invention contains a first antibody which recognizes the amino acid sequence of SEQ ID NO: 2 that is present in the whole amino acid sequence of NS1. As a result, a cross-reaction with a virus or the like other than dengue virus can be inhibited, and dengue virus can be detected rapidly and specifically. The amino acid sequence of SEQ ID NO: 2 corresponds to the amino acid sequence of from the 100th to 121st residues of the whole amino acid sequence of NS1 of SEQ ID NO: 1.

That the first antibody “recognizes the amino acid sequence of SEQ ID NO: 2 that is present in the whole amino acid sequence of dengue virus NS1 of SEQ ID NO: 1” specifically means that, for example, when the competitive inhibition test by ELISA method (the competitive inhibition ELISA test) described below in the Examples is conducted using the whole length dengue virus NS1 and the peptide having the amino acid sequence of SEQ ID NO: 2, the reaction of the antibody to dengue virus NS1 is competitively inhibited by the presence of the peptide having the amino acid sequence of SEQ ID NO: 2. To improve the detection sensitivity and the specificity, the absorbance in the competitive inhibition ELISA test described below in the Examples is preferably 50% or less, more preferably 25% or less, further preferably 12.5% or less, particularly preferably 10% or less, most preferably 5% or less, where the absorbance of the case in which the peptide having the amino acid sequence of SEQ ID NO: 2 is not added (control) is considered 100%.

The first antibody preferably does not substantially recognize the amino acid sequences of SEQ ID NOs: 3 to 8 that are present in the whole amino acid sequence of NS1. This can further improve the specificity and the detection sensitivity. In the invention, that an antibody “does not substantially recognize an amino acid sequence” specifically means, for example, that the absorbance in the competitive inhibition ELISA test described below in Reference Example 1 is within −25% of that of the control.

The detection part in the immunochromatography analysis device of the invention contains a second antibody which recognizes the three-dimensional structure of NS1. The NS1 that the second antibody recognizes may be NS1 which maintains a sufficient three-dimensional structure for maintaining the antigen-antibody reaction with at least a certain antibody, of the three-dimensional structure of naturally existing dengue virus NS1, and those which no longer maintain the substantial antigen-antibody reaction of NS1 to the antibody after the three-dimensional structure of naturally existing NS1 has been destroyed by SDS-PAGE (SDS-polyacrylamide gel electrophoresis) or the like are excluded.

The second antibody may be an antibody which does not substantially undergo the antigen-antibody reaction with the protein in which the three-dimensional structure of the site for the antigen-antibody reaction in naturally existing NS1 has been destroyed. An example of such an antibody is an antibody which does not undergo the antigen-antibody reaction with the whole length dengue virus NS1 separated by SDS-PAGE by western blotting. That is, an embodiment of the second antibody is an antibody which undergoes the antigen-antibody reaction with naturally existing (native) NS1 and which does not undergo the antigen-antibody reaction with NS1 separated by SDS-PAGE by western blotting. In the invention, that an antibody “does not undergo the antigen-antibody reaction” means that the antibody does not substantially undergo the antigen-antibody reaction and means that the antibody does not undergo the antigen-antibody reaction at a detectable level in the immunoassay or does not specifically react.

In the invention, that an antibody “does not undergo the antigen-antibody reaction by western blotting” means that the antibody does not undergo the antigen-antibody reaction at a detectable level under the standard conditions for the antibody concentration, the antigen concentration, the substrate concentration, the reaction period or the like in western blotting.

That the second antibody does not undergo the antigen-antibody reaction with the whole length dengue virus NS1 separated by SDS-PAGE by western blotting can be confirmed, for example using a commercial antibody which has been confirmed to undergo the antigen-antibody reaction with dengue virus NS1 by western blotting as a positive control antibody. Specifically, for example, that dengue virus NS1 cannot be detected under the condition under which the positive control antibody undergoes the antigen-antibody reaction with dengue virus NS1 on a polyvinylidene difluoride (PVDF) membrane and can detect NS1 can be used as a standard.

“SDS-PAGE” in the invention is a separation/analysis method of a protein which is commonly used in the technical field to which the invention pertains and can be typically conducted according to the method of Laemmli, U. K. (Nature, 227: 680-685 (1970)) although SDS-PAGE is not limited to the method.

Specifically, for example, SDS-PAGE can be conducted by the following procedures. First, a resolving gel containing polyacrylamide at a concentration of 10 to 15% is cast between gel plates, and a stacking gel containing 3 to 5% polyacrylamide is cast on top of the resolving gel. The produced gels are mount in a slab electrophoresis apparatus. To a solution containing dengue virus NS1, an equivalent amount of 2× sample buffer (125 mM Tris-HCl, 20% glycerol, 2% SDS, 2% 2-mercaptoethanol, 0.001% bromophenol blue, pH 6.8) is added, and the mixture is heat-treated at 100° C. for 5 to 10 minutes. The sample for electrophoresis is thus prepared. The sample for electrophoresis and a commercial molecular weight marker are loaded in the lanes created in the stacking gel, and the electrophoresis is conducted using an electrophoresis buffer (192 mM glycine, 0.1% SDS, 24 mM Tris, pH 8.3) with a constant current of 20 mA for 30 to 90 minutes. The whole length dengue virus NS1 separated by SDS-PAGE can be obtained in a band corresponding to the molecular weight of about 42 kDa in the resolving gel.

The solution containing dengue virus NS1 used for SDS-PAGE is not limited as long as the solution contains dengue virus NS1 in an amount sufficient for the antigen-antibody reaction with the antibody in the eventual western blotting, such as 1 to 2 mg, and the solution may be purified but does not have to be purified in terms of NS1. Examples of the solution containing dengue virus NS1 include a dengue virus suspension, a commercial dengue vaccine, a recombinant dengue virus NS1 solution and the like.

SDS in the 2× sample buffer used for SDS-PAGE can be used with an appropriate modification in a concentration range of 0.5 to 5% by weight depending on the amount of dengue virus NS1 considering that the amount of bound SDS is about 1.2 to 1.5 based on the protein amount considered as 1. Moreover, 2-mercaptoethanol in the 2× sample buffer functions as a reducing agent which cuts the disulfide bonds in dengue virus NS1 and may be used with an appropriate modification in a concentration range of 1 to 10% by weight. A reducing agent composed of another substance such as dithiothreitol (DTT) can also be used.

“Western blotting” in the invention can be conducted by transferring the whole length dengue virus NS1 separated by SDS-PAGE onto a PVDF membrane according to, for example, the method of Towbin H. et al. [Proc.Natl.Acad.Sci. U.S.A., 76: 4350-4354 (1979)] but is not limited to this method.

Specifically, for example, a PVDF membrane is immersed in 100% methanol for 10 seconds and in an electrode buffer for transfer (192 mM glycine, 5% methanol, 25 mM Tris-HCl, pH 8.3) for 30 minutes and used for the transfer. The transfer apparatus is assembled by placing a filter paper, the PVDF membrane, the gel after the completion of SDS-PAGE and a filter paper in this order on the anode plate and fixing the cathode plate on top. The filter papers are immersed in advance in the electrode buffer for transfer for two to three minutes.

The transfer is conducted with a constant current of 1.9 mA/cm² for 60 to 90 minutes. The PVDF membrane after the completion of the transfer is incubated in a blocking solution [0.5% bovine serum albumin (BSA), 10 mM Tris-HCl, 140 mM NaCl, 0.01% Tween 20, pH 7.5] at room temperature for 60 minutes for blocking operation. After the completion of the blocking, the membrane is incubated and washed twice with a wash buffer (10 mM Tris-HCl, 140 mM NaCl, 0.01% Tween 20, pH 7.5) for five minutes and incubated and reacted using the second antibody (dengue virus NS1 antibody) as a primary antibody at room temperature for 90 minutes.

After the completion of the reaction with the primary antibody, the membrane is incubated and washed twice with the wash buffer for five minutes and incubated and reacted using an antibody which specifically reacts with the primary antibody which is labeled with a labeling substance such as an enzyme, a fluorescent substance and a radioisotope as a secondary antibody at room temperature for 60 minutes. After the completion of the reaction with the secondary antibody, the membrane is incubated and washed twice with the wash buffer for five minutes, and then the primary antibody bound to the dengue virus NS1 transferred to the PVDF membrane is visualized using the properties of the labeling substance for the detection of western blotting.

To improve the detection sensitivity and the specificity, the second antibody is preferably an antibody which does not undergo the antigen-antibody reaction with dengue virus NS1 at the same antibody concentration as the antibody concentration at which the positive control antibody can detect dengue virus NS1 by western blotting, more preferably an antibody which does not react with dengue virus NS1 at the antibody concentration which is two times higher, further preferably an antibody which does not undergo the antigen-antibody reaction with dengue virus NS1 at an antibody concentration which is five times or 10 times higher.

To improve the detection sensitivity and the specificity, the second antibody is preferably an antibody which does not undergo the antigen-antibody reaction with dengue virus NS1 at the same concentration as the antigen concentration of dengue virus NS1 which the positive control antibody can detect by western blotting, more preferably an antibody which does not react at the antigen concentration which is two times higher, further preferably an antibody which does not undergo the antigen-antibody reaction with dengue virus NS1 at a concentration which is five times or 10 times higher.

The second antibody preferably recognizes a polypeptide containing the whole amino acid sequence of dengue virus NS1 of SEQ ID NO: 1 and does not substantially recognize the amino acid sequences of SEQ ID NOs: 2 to 8 that are present in the whole amino acid sequence of dengue virus NS1. This can further improve the detection sensitivity to dengue virus.

Because the labeling substance-holding part and the detection part contain the respective antibodies, the immunochromatography analysis device of the invention can reduce a cross-reaction with, for example, Zika virus, Japanese encephalitis virus or the like, which belongs to Flaviviridae like dengue virus.

In the immunochromatography analysis device of the invention, the detection part preferably further contains an antibody which recognizes the amino acid sequence of SEQ ID NO: 2 that is present in the whole amino acid sequence of NS1 (the first antibody) in addition to the second antibody. This can further reduce a cross-reaction with an antigen other than dengue virus and at the same time improve the sensitivity to dengue virus. When the detection part contains the first antibody in addition to the second antibody, the ratio (mass ratio) of the first antibody and the second antibody is generally preferably 1:9 to 9:1 to improve the detection sensitivity.

Examples of the first antibody and the second antibody include natural antibodies such as polyclonal antibodies and monoclonal antibodies, chimeric antibodies, humanized antibodies and single-chain antibodies that can be produced using gene recombination, human antibodies that can be produced using a human antibody-producing transgenic animal or the like, antibodies produced by phage display and fragments thereof with binding capacity. Monoclonal antibodies are preferable to improve the sensitivity.

The first antibody and the second antibody used in the invention can be produced using a dengue virus NS1 peptide as an immunogen by administering the peptide to the antibody-producing animal species. Examples of the method for producing an antibody that recognizes dengue virus NS1 are explained below.

Regarding the antibody-producing animal species, for example, human, mouse, rat, rabbit, goat, horse and the like can be used. The immunoglobulin may be any of IgG, IgM, IgA, IgE and IgD.

In an embodiment of the method for producing the first antibody, the dengue virus NS1 peptide as the immunogen can be produced by a known general production method. That is, dengue virus NS1 peptide that is extracted and purified from dengue virus, dengue virus NS1 peptide that is obtained by expressing cloned gene of dengue virus NS1 in a host such as Escherichia coli by genetic engineering and extracting and purifying the peptide or a polypeptide which composes a part of dengue virus NS1 peptide can be used as the immunogen.

In an embodiment of the method for producing the second antibody, the immunogen is preferably dengue virus NS1 which has not been treated with a sample buffer for SDS-PAGE containing a reducing agent, more preferably dengue virus NS1 which has not been treated with SDS, which is an anionic surfactant. Preferable examples of the immunogen for the second antibody include a suspension of dengue virus in a buffer containing no anionic surfactant, the whole length dengue virus NS1 and the like.

With respect to a monoclonal antibody, according to a general method, after hybridizing spleen cells of a mouse immunized with the immunogen and myeloma cells, a hybridoma that produces the target antibody is selected, and the monoclonal antibody produced by the hybridoma is obtained [for example, see the Köhler and Milstein's technique (Nature 256 (1975) 495-497)]. A polyclonal antibody is obtained by separating the target antibody from the antiserum obtained by immunizing an animal for production (for example, human, mouse, rat, rabbit, goat, horse or the like) with the immunogen according to a general method.

Screening to obtain the hybridoma clone that produces a monoclonal antibody can be conducted by culturing hybridomas for example in a microtiter plate and measuring the reactivities of the culture supernatants of the wells in which the growth is observed with the immunogen by enzyme immunoassay such as ELISA method.

The hybridoma can be cultured using a medium (for example, DMEM containing 10% fetal bovine serum), and the supernatant of the culture solution obtained by centrifugation can be used as a monoclonal antibody solution. Also, ascites can be caused by injecting the hybridoma into the abdominal cavity of the origin animal, and the obtained ascites can be used as a monoclonal antibody solution. The monoclonal antibody is preferably isolated and/or purified. In this manner, an antibody that recognizes dengue virus NS1 can be produced.

Of antibodies which recognize dengue virus NS1, the first antibody, which is an antibody that recognizes the amino acid sequence of SEQ ID NO: 2, can be obtained, for example, by the following method and used. By subjecting to an ELISA test, western blotting or the like using a peptide fragment corresponding to the amino acid sequence of SEQ ID NO: 2, a hybridoma which produces an antibody exhibiting stronger reactivity with the amino acid sequence is selected from the hybridomas that produce antibodies recognizing dengue virus NS1.

Of antibodies which recognize dengue virus NS1, the second antibody, which is an antibody that recognizes the three-dimensional structure of dengue virus NS1, can be obtained and used, for example by selecting a hybridoma which produces an antibody that does not undergo the antigen-antibody reaction with the whole length dengue virus NS1 separated by SDS-PAGE by western blotting from the hybridomas that produce antibodies recognizing dengue virus NS1.

The first antibody and the second antibody can be prepared by culturing the respective hybridomas in a medium which is usually used for cell culture and collecting from the culture supernatants. Moreover, the antibodies can also be prepared by accumulating ascites by injecting the hybridomas into the abdominal cavities of the origin animals and collecting from the ascites.

Next, an embodiment of the immunochromatography analysis device of the invention is explained referring to the drawings.

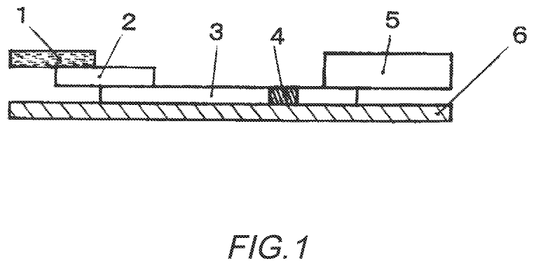

In an embodiment, the immunochromatography analysis device of the invention is composed of a sample application part (1), a labeling substance-holding part (2), a chromatography medium part (3), a detection part (4), an absorption part (5) and a backing sheet (6) as shown in FIG. 1.

The sample application part (1) is a part in the immunochromatography analysis device to which a sample containing an analyte is applied. The sample application part (1) can be composed of a porous sheet having the properties of rapidly absorbing the sample but allowing the sample to move rapidly. Examples of the porous sheet include cellulose filter paper, glass fibers, polyurethane, polyacetate, cellulose acetate, nylon, cotton cloth and the like.

The labeling substance-holding part (2) holds a labeled antibody labeled with a labeling substance described below (also simply called the labeled antibody below) and is a part in which the labeled antibody binds to the substance to be detected in the analyte. The labeled antibody is the first antibody which recognizes the amino acid sequence of SEQ ID NO: 2 that is present in the whole amino acid sequence of dengue virus NS1, and the first antibody binds to the dengue virus NS1 in the analyte when the sample moves in the labeling substance-holding part (2).

For the labeling substance-holding part (2), a membrane of glass fibers, cellulose or the like is usually used.

The amount of the labeled antibody in the labeling substance-holding part (2) is usually 0.05 μg/device to 0.5 μg/device, preferably 0.05 μg/device to 0.25 μg/device, more preferably 0.07 μg/device to 0.1 μg/device. When the amount of the labeled antibody in the labeling substance-holding part (2) is in the range, an excellent detection sensitivity can be achieved.

The amount of the labeled antibody per unit area of the labeling substance-holding part (2) is usually 0.05 μg/cm² to 1.0 μg/cm², preferably 0.1 μg/cm² to 0.8 μg/cm², more preferably 0.17 μg/cm² to 0.6 μg/cm². When the amount of the labeled antibody per unit area of the labeling substance-holding part (2) is in the range, an excellent detection sensitivity can be achieved.

An enzyme or the like is also generally used as the labeling substance for labeling an antibody in an immunochromatography analysis, but an insoluble carrier is preferably used as the labeling substance because the insoluble carrier is suitable for visually determining the presence of the substance to be detected. That is, in the invention, a labeled antibody which is labeled by sensitizing the first antibody by an insoluble carrier is preferably used as the antibody contained in the labeling substance-holding part (2). In this regard, the means for sensitizing the first antibody by the insoluble carrier may be in accordance with a known method.

As the insoluble carrier used as the labeling substance, particles of a metal such as gold, silver and platinum, particles of a metal oxide such as iron oxide, particles of a nonmetal such as sulfur, latex particles of a synthetic polymer or other insoluble carriers can be used. As described above, the insoluble carrier is a labeling substance which is suitable for visually determining the presence of the substance to be detected and preferably has a color to make the visual determination easy. Metal particles and metal oxide particles themselves have peculiar natural colors according to the particle diameter, and the colors can be used as labels.

The insoluble carrier used as the labeling substance is especially preferably gold particles because gold particles are simple to detect and do not easily cohere and because nonspecific color development is unlikely to occur. To improve the detection sensitivity, the average particle diameter of the gold particles is, for example, 10 nm to 250 nm, preferably 35 nm to 120 nm. The average particle diameter can be calculated by measuring the projected area circle equivalent diameters of 100 particles at random using projected pictures taken with a transmission electron microscope (TEM: manufactured by JEOL Ltd., JEM-2010) and calculating from the average.

The amount of the gold particles in the labeling substance-holding part is, per unit area of the labeling substance-holding part, usually 0.006 μg/cm² to 0.42 μg/cm², preferably 0.01 μg/cm² to 0.3 μg/cm², more preferably 0.01 μg/cm² to 0.2 μg/cm². This is because, by determining the amount in the range, the labeled particles can be developed while the particles are dispersed, and the recognition sites for the antibody are not inhibited, resulting in an increase in the sensitivity.

The chromatography medium part (3) is a part for development of chromatography. The chromatography medium part (3) is an inert membrane composed of a fine porous substance which causes a capillary phenomenon. For example, membranes made of nitrocellulose (also called nitrocellulose membranes below) and membranes made of cellulose acetate (also called cellulose acetate membranes below) are preferable, and nitrocellulose membranes are further preferable, because the membranes do not have the property of reacting with the detection reagent or the immobilizing reagent used for chromatography or with the substance to be detected or the like and because the effects of the invention are enhanced. In this regard, cellulose membranes, nylon membranes and porous plastic clothes (for example, polyethylene, polypropylene or the like) can also be used.

The nitrocellulose membranes may be any nitrocellulose membranes as long as the membranes mainly contain nitrocellulose, and membranes containing nitrocellulose as the main material, such as a pure product or a nitrocellulose-mixed product, can be used.

The nitrocellulose membranes can also contain a substance which further enhances the capillary phenomenon. The substance is preferably a substance which weakens the surface tension of the membrane surface and attains hydrophilicity. For example, a substance which has amphipathic action, like saccharides, derivatives of amino acids, fatty acid esters, various synthetic surfactants, alcohols or the like, and which does not affect the movement of the substance to be detected and does not affect the color development of the labeling substance is preferable.

The nitrocellulose membranes are porous and cause a capillary phenomenon. The indicator of the capillary phenomenon can be confirmed by measuring the speed of water absorption (water absorption time: capillary flow time). The speed of water absorption affects the detection sensitivity and the test time.

The form and the size of the chromatography medium part (3), which is typically any of the nitrocellulose membranes or the cellulose acetate membranes described above, are not particularly limited and may be any form and any size as long as they are appropriate for the actual operation and for the observation of the reaction results.

In order to further make the operation simpler, a support composed of plastic or the like is preferably provided on the back surface of the chromatography medium part (3). The properties and state of the support are not particularly limited. When the measurement results are observed by a visual evaluation, the support is preferably a support having a color which is not similar to the color achieved by the labeling substance, and the support is usually preferably colorless or white.

In order to prevent the deterioration of the analysis accuracy due to nonspecific adsorption on the chromatography medium part (3), the chromatography medium part (3) can be subjected to blocking treatment by a known method according to the need. For the blocking treatment, in general, a protein such as bovine serum albumin, skim milk, casein and gelatin is preferably used. After the blocking treatment, the chromatography medium part (3) may be washed with one or a combination of two or more of surfactants such as Tween 20, Triton X-100 and SDS according to the need.

The detection part (4) is formed at any position on the chromatography medium part (3) and contains the second antibody which recognizes the three-dimensional structure of dengue virus NS1. The second antibody can be immobilized on the detection part (4) according to a general method.

In the detection part (4), dengue virus in the analyte that has passed through on the chromatography medium part as the mobile phase specifically reacts and binds in a manner that the dengue virus is sandwiched between the second antibody that is immobilized on the detection part (4) and the first antibody to which the labeling substance is bound.

The amount of the second antibody contained in the detection part (4) is usually 0.1 μg to 3.0 μg, preferably 0.3 μg to 2.0 μg, more preferably 0.3 μg to 1.0 μg. When the amount of the second antibody contained in the detection part (4) is in the range, an excellent detection sensitivity can be achieved.

The amount of the second antibody per unit area of the detection part (4) is usually 0.04 μg/cm² to 1.0 μg/cm², preferably 0.125 μg/cm² to 0.8 μg/cm², more preferably 0.125 μg/cm² to 0.42 μg/cm². When the amount of the second antibody per unit area of the detection part (4) is in the range, an excellent detection sensitivity can be achieved.

The absorption part (5) is provided at the end of the chromatography medium part (3) to absorb liquids such as the analyte and the development solution which have passed through the detection part (4). In the invention, for example, glass fibers, pulp, cellulose fibers or these nonwoven clothes to which a polymer such as acrylic polymers and a hydrophilic agent having an ethylene oxide group or the like have been added are used for the absorption part (5), and glass fibers are particularly preferable.

The backing sheet (6) is a base material. One surface thereof is adhesive because an adhesive is applied on the surface or an adhesive tape is attached, and the sample application part (1), the labeling substance-holding part (2), the chromatography medium part (3), the detection part (4) and the absorption part (5) are partially or entirely closely adhered and provided on the adhesive surface. The base material is not particularly limited as long as the backing sheet (6) is not permeable or breathable with respect to the sample solution due to the adhesive.

The immunochromatography analysis device of the invention is usually subjected to drying treatment before being finished as a product. The drying temperature is, for example, 20° C. to 50° C., and the drying time is 0.5 hours to 1 hour.

In the immunochromatography analysis device of the invention, the labeling substance-holding part contains the first antibody which recognizes the amino acid sequence of SEQ ID NO: 2 that is present in the whole amino acid sequence of dengue virus NS1, and the detection part contains the second antibody which recognizes the three-dimensional structure of dengue virus NS1. As a result, a cross-reaction with another antigen can be reduced more, and dengue virus can be detected more specifically.

<Immunochromatography Analysis Kit>

The immunochromatography analysis kit of the invention includes the immunochromatography analysis device and an analyte dilution solution for diluting and developing an analyte.

In the immunochromatography analysis kit of the invention, the analyte dilution solution can be used also as a development solution. Water is usually used as a solvent of the analyte dilution solution, and a buffer solution, a salt and a nonionic surfactant are contained. A kind or two or more kinds of a protein, a polymer compound (such as PVP), an ionic surfactant or a polyanion for, for example, promoting the antigen-antibody reaction or inhibiting a nonspecific reaction, an antibacterial agent, a chelating agent and the like may be further added.

When the analyte dilution solution is used as a development solution, the analyte and the development solution can be mixed in advance and then supplied/dropped as the sample to the sample application part for development, or the development solution may be supplied/dropped to the sample application part for development after supplying/dropping the sample containing the analyte to the sample application part in advance.

<Immunochromatography Analysis Method>

The immunochromatography analysis method of the invention includes the following steps (1) to (4), and dengue virus contained in an analyte is detected using the immunochromatography analysis kit.

(1) A step of applying an analyte-containing solution obtained by diluting the analyte with the analyte dilution solution as a sample to the sample application part

(2) A step of causing the first antibody which recognizes the amino acid sequence of SEQ ID NO: 2 that is present in the whole amino acid sequence of dengue virus NS1 and which is held in the labeling substance-holding part to recognize dengue virus in the analyte

(3) A step of developing the analyte and the first antibody as a mobile phase on the chromatography medium part

(4) A step of detecting the dengue virus in the developed mobile phase with the second antibody which recognizes the three-dimensional structure of dengue virus NS1 contained in the detection part

Each step is explained below.

(1) A step of applying an analyte-containing solution obtained by diluting the analyte with the analyte dilution solution as a sample to the sample application part

In the step (1), first, an analyte-containing solution is preferably obtained by adjusting or diluting the analyte with the analyte dilution solution to a concentration at which the analyte moves smoothly in the immunochromatography medium without deteriorating the measurement accuracy. Those described above can be used as the analyte dilution solution. Secondly, a certain amount (usually 0.1 ml to 2 ml) of the analyte-containing solution is dropped as a sample onto the sample application part (1). When the sample is dropped to the sample application part (1), the sample starts to move in the sample application part (1).

The analyte used in the invention is an analyte that may contain dengue virus, which is the substance to be detected, as described above. Specific examples include serum, plasma, whole blood, semen, spinal fluid or the like of a patient infected with dengue virus, but the analyte is not limited to these examples.

(2) A step of causing the first antibody which recognizes the amino acid sequence of SEQ ID NO: 2 that is present in the whole amino acid sequence of dengue virus NS1 and which is held in the labeling substance-holding part to recognize dengue virus in the analyte

The step (2) is a step for transferring the sample applied to the sample application part in the step (1) to the labeling substance-holding part (2) and causing the first antibody to which the labeling substance is bound and which is held in the labeling substance-holding part to recognize dengue virus, which is the substance to be detected, in the analyte. Those described above can be used as the labeling substance.

(3) A step of developing the analyte and the first antibody as a mobile phase on the chromatography medium part

The step (3) is a step in which, after dengue virus, which is the substance to be detected, has been recognized by the first antibody to which the labeling substance is bound in the labeling substance-holding part in the step (2), the analyte and the first antibody are caused to pass through on the chromatography medium part as a mobile phase.

(4) A step of detecting the dengue virus in the developed mobile phase with the second antibody which recognizes the three-dimensional structure of dengue virus NS1 contained in the detection part

The step (4) is a step in which the dengue virus in the analyte that has passed through on the chromatography medium part as the mobile phase specifically reacts and binds by a specific antigen-antibody binding reaction in a manner that the dengue virus is sandwiched between the second antibody that is immobilized on the detection part and the first antibody to which the labeling substance has bound in the step (2), resulting in the coloration of the detection part.

When dengue virus, which is the substance to be detected, is absent, the labeling reagent dissolved in the water content of the sample does not cause the specific binding reaction even when the labeling reagent passes through the detection part on the chromatography medium part, and thus the detection part is not colored.

At the end, the water content in the analyte-containing solution moves to the absorption part (5).

### EXAMPLES

The invention is further explained below with Examples, but the invention is not limited to the following examples.

### Production Example 1 (Production of Antibodies)

Antibodies which recognize dengue virus NS1 were produced as follows. First, a DNA encoding the amino acid sequence of dengue virus NS1 of SEQ ID NO: 1 was synthesized. A His-tag expression vector, pET302/NT-His, was cut with a restriction enzyme, EcoRI, then treated with alkaline phosphatase as dephosphorylation treatment and mixed with the DNA, and ligation reaction was caused using DNA Ligation Kit Ver. 2 (Takara Bio Inc.).

The recombinant NS1 plasmid to which the target gene had been incorporated was introduced into a recombinant protein expression host, E. coli BL(DE3)pLysS (Novagen). The transformed bacterium was cultured on an LB agar plate, and a colony obtained was cultured with LB liquid medium. The expression of the recombinant NS1 was induced by adding 1 mM IPTG (Takara Bio Inc.), and then E. coli was collected. The collected bacterium was suspended again in a solubilization buffer [0.5% Triton X-100 (sigma), 10 mM imidazole, 20 mM phosphate and 0.5 M NaCl (pH 7.4) (Amersham)] and solubilized by ultrasonic treatment, and the recombinant NS1 was purified using His trap Kit (Amersham). The purified protein was dialyzed using phosphate-buffered saline (referred to as PBS below), and the target recombinant NS1 was thus obtained.

Monoclonal antibodies to the recombinant NS1 were produced using the obtained recombinant NS1 as the antigen for immunization. The monoclonal antibodies were produced as follows according to a general method. The recombinant NS1 in an amount of 100 μg and an equivalent amount of Adjuvant Complete Freund (Difco) were mixed, and a mouse (BALB/c, five weeks old, Japan SLC, Inc.) was immunized three times. The spleen cells were used for cell fusion. Mouse myeloma cells, Sp2/0-Ag14 cells (Shulman et al., 1978) were used for the cell fusion. A culture solution obtained by adding 0.3 mg/ml L-glutamine, 100 U/ml penicillin G potassium, 100 μg/ml streptomycin sulfate and 40 μg/ml Gentacin to Dulbecco's Modified Eagle Medium (Gibco) (DMEM) and further adding fetal bovine serum (JRH) at 10% was used for culturing the cells. The cells were fused by mixing the spleen cells of the immunized mouse and Sp2/0-Ag14 cells and adding polyethylene glycol solution (Sigma) thereto. The fused cells were cultured in HAT-DMEM [serum-containing DMEM containing 0.1 mM sodium hypoxanthine, 0.4 μM aminopterin and 0.016 mM thymidine (Gibco)], and the production of antibodies in the culture supernatant was confirmed by enzyme-linked immunosorbent assay (ELISA method). Antibody production-positive cells were cultured in HT-DMEM [serum-containing DMEM containing 0.1 mM sodium hypoxanthine and 0.16 mM thymidine] and further cultured in serum-containing DMEM.

The cloned cells were injected into the abdominal cavities of mice (BALB/c, retired, Japan SLC, Inc.) to which 2,6,10,14-tetramethylpentadecane (Sigma) had been injected, and the ascites was collected. The ascites was subjected to a protein G column, and monoclonal antibodies were purified.

The monoclonal antibodies thus obtained were screened by direct ELISA method using a 96-well plate in which the recombinant NS1 was immobilized. As a result, four types of antibody recognizing dengue virus NS1 were obtained. The four types of antibody are called antibodies of No. 1 to No. 4 in the following explanation.

### Reference Example 1 (Competitive Inhibition ELISA Test)

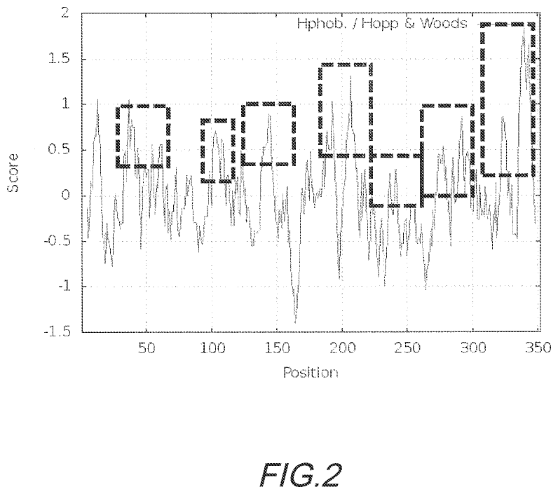

By a hydrophilicity plot and alignment of NS1s of DENV-1 to DENV-4, the amino acid sequences of SEQ ID NOs: 2 to 8 having high hydrophilicity and high homology were identified. The hydrophilicity plot was created by calculating dengue virus NS1s according to the Hopp & Woods rules using ExPASy. The results of the hydrophilicity plot are shown in FIG. 2.

The amino acid sequences of SEQ ID NOs: 2 to 8 were obtained as described above based on the information on the amino acid sequences of the NS1 regions obtained from the sequence information registered at the National Center for Biotechnology Information, U.S. (NCBI) [DENV-1 NCBI Accession No. AAA42940 (777 . . . 1130), DENV-2 NCBI Accession No. ACW82881 (777 . . . 1130), DENV-3 NCBI Accession No. YP_001621843 (774 . . . 1125), DENV-4 NCBI Accession No. NP_073286 <775 . . . 1126>] and the like. For the analysis of homology, Molecular evolutionary genetics analysis was used.

Whether or not the antibodies recognizing dengue virus NS1 produced in Production Example 1 (the antibodies of No. 1 to No. 4) recognize the amino acid sequences of SEQ ID NOs: 2 to 8 was examined by a competitive inhibition ELISA test. The peptides used for the competitive inhibition ELISA test were seven types of peptide having the amino acid sequences of SEQ ID NOs: 2 to 8 (peptides 1 to 7 shown below) and were produced by solid-phase peptide synthesis, which is a general method for chemically synthesizing a peptide.

Peptide 1:
(SEQ ID NO: 3)
ELKCGSGIFVTNEVHTWTEQYKFQ
Peptide 2:
(SEQ ID NO: 2)
GKKMIRPQPMEHKYSWKSWGKA
Peptide 3:
(SEQ ID NO: 4)
IDGPNTPECPDNQRAWN
Peptide 4:
(SEQ ID NO: 5)
AVHADMGYWIESEKNETWKLARASFIEVKT
Peptide 5:
(SEQ ID NO: 6)
GGPISQHNYRPGYFTQTAGPWHLG
Peptide 6:
(SEQ ID NO: 7)
GTTVVVDEHCGNRGPSLRTTTVTGK
Peptide 7:
(SEQ ID NO: 8)
GEDGCWYGMEIRPVKEKEENLVKSMVSA

The recombinant NS1 produced in Production Example 1 was diluted with PBS to a concentration of 5 μg/mL, then dispensed to a 96-well plate for ELISA (Nunc Immuno modules, manufactured by Thermo Fisher Scientific, code 469949) at 100 μL/well and left to stand still at 4° C. overnight. After washing each well three times with 0.05% Tween 20-containing PBS (called “PBST” below) (400 μL/well), 1% BSA-containing PBST (called “BSA-PBST” below) was added at 100 μL/well, and blocking was conducted by leaving the plate to stand still at room temperature for an hour.

After washing three times with PBST, 100 μL of a 50% blocking solution containing a primary antibody solution of No. 1 to No. 4 at 20 μg/mL and a peptide (of the peptides 1 to 7) at 5 mg/mL was added to the wells, and the plate was incubated at 37° C. for an hour.

The primary antibody solutions were removed, and the wells were washed three times with 300 μL of PBST (0.05% Tween 20 in PBS). The liquids remaining in the wells were removed by hitting the plate onto a paper towel.

As the secondary antibody, 100 μL of 1 μg/mL Anti Mouse IgG (H+L), Rabbit, IgG Whole, Peroxidase Conjugated (manufactured by Wako Pure Chemical Industries, Ltd., code 014-17611) was added to the wells, and the plate was incubated at 37° C. for 1.5 hours. Then, the secondary antibody solution was removed, and the wells were washed three times with 300 μL of PBST (0.05% Tween 20 in PBS). The liquids remaining in the wells were removed by hitting the plate onto a paper towel.

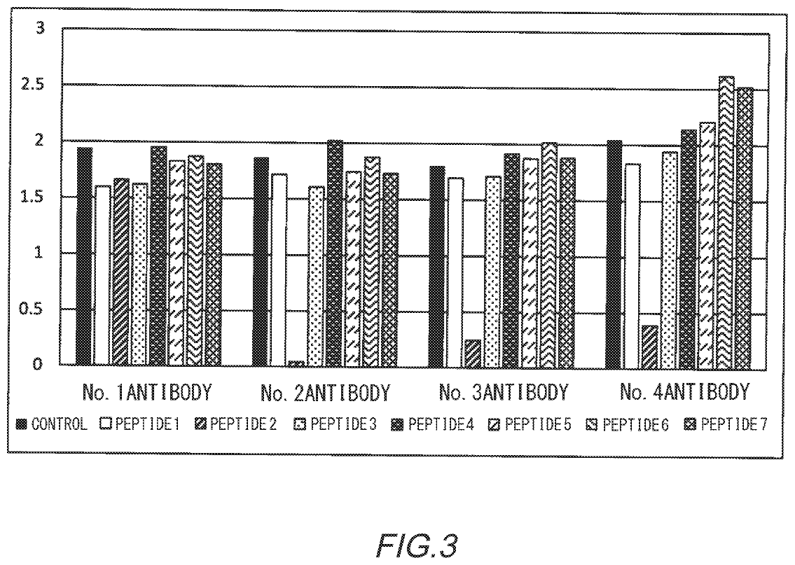

Sure Blue Reserve TMB Microwell Peroxidase Substrate (1-Component) (manufactured by KPL, code 53-00-01) in a volume of 100 μL was added to the wells as a chromogenic substrate, and the reaction was advanced for 15 minutes. The reaction was stopped by adding 100 μL of 2N sulfuric acid. Then, the absorbances at 450 nm were measured using a microplate reader (manufactured by BIORAD). The results are shown in Table 1 and FIG. 3.

TABLE 1

| | | Peptide | Peptide | Peptide | Peptide | Peptide | Peptide | Peptide |
|---|---|---|---|---|---|---|---|---|
| | Control | 1 | 2 | 3 | 4 | 5 | 6 | 7 |
| No. 1 | 1.936 | 1.597 | 1.663 | 1.622 | 1.95 | 1.83 | 1.872 | 1.805 |
| Antibody | | | | | | | | |
| No. 2 | 1.861 | 1.714 | 0.045 | 1.609 | 2.017 | 1.743 | 1.875 | 1.729 |
| Antibody | | | | | | | | |
| No. 3 | 1.797 | 1.693 | 0.249 | 1.711 | 1.913 | 1.872 | 2.014 | 1.877 |
| Antibody | | | | | | | | |
| No. 4 | 2.038 | 1.832 | 0.392 | 1.938 | 2.139 | 2.204 | 2.616 | 2.519 |
| Antibody | | | | | | | | |

As shown in Table 1 and FIG. 3, it was found that the antibody of No. 1 does not recognize any of the amino acid sequences of SEQ ID NOs: 2 to 8. Moreover, it was found that the antibodies of No. 2 to No. 4 recognize the amino acid sequence of SEQ ID NO: 2.

### Reference Example 2 (Dot Blotting)

The recombinant NS1 (DENV-4) was spotted on a nitrocellulose membrane at 0 ng/well, 150 ng/well, 300 ng/well or 600 ng/well, and then blocking reaction was advanced in 30 μL/well of 1% BSA-containing PBS at room temperature for 20 minutes.

Reaction with the antibodies of No. 1 to No. 4 which were each adjusted at 20 μg/ml was advanced at room temperature for an hour, and the membrane was moved to a petri dish and then washed three times with 3 mL of PBS-T. The wash solution was removed completely, and 2 mL of 1 μg/mL Anti Mouse IgG (H+L), Rabbit, IgG Whole, Peroxidase Conjugated (manufactured by Wako Pure Chemical Industries, Ltd., code 014-17611) as the secondary antibody was added to the petri dish. The membrane was incubated at room temperature for 1.5 hours.

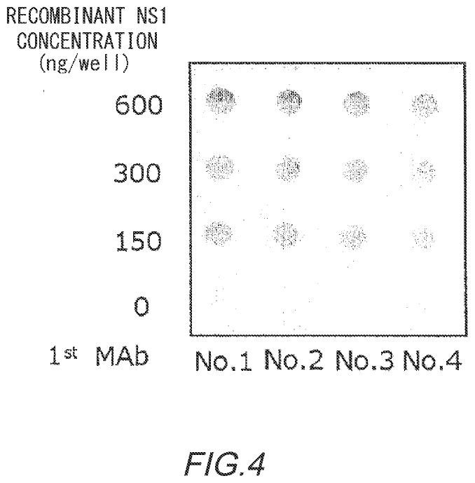

Then, 1 mL of TMB Solution for Western Blotting (Nacalai Tesque, Inc.) as the substrate was added for wetting gradually for two minutes, and the color was developed. The results are shown in FIG. 4.

As shown in FIG. 4, it was found that the antibodies of No. 1 to No. 4 all exhibit the antigen-antibody reaction with the recombinant NS1.

### Reference Example 3 (Western Blotting)

A resolving gel containing polyacrylamide at a concentration of 10% was cast between gel plates, and a stacking gel containing 35% polyacrylamide was cast on top of the resolving gel. The produced gels were mount in slab electrophoresis apparatus. To solutions containing dengue virus NS1 (0.25 μg of DENV1 NS1 or DENV2 NS1 per lane), an equivalent amount of 2× sample buffer (125 mM Tris-HCl, 20% glycerol, 2% SDS, 2% 2-mercaptoethanol, 0.001% bromophenol blue, pH 6.8) was added, and the mixtures were heat-treated at 100° C. for five minutes. The samples for electrophoresis were thus prepared. The samples for electrophoresis and a commercial molecular weight marker were loaded in the lanes created in the stacking gels, and the electrophoresis was conducted using an electrophoresis buffer (192 mM glycine, 0.1% SDS, 24 mM Tris, pH 8.3) with a constant current of 20 mA for 30 minutes.

PVDF membranes were immersed in 100% methanol for 10 seconds and in an electrode buffer for transfer (192 mM glycine, 5% methanol, 25 mM Tris-HCl, pH 8.3) for 30 minutes and used for the transfer. A transfer apparatus was assembled by placing a filter paper, a PVDF membrane, a gel after the completion of SDS-PAGE and a filter paper in this order on the anode plate and fixing the cathode plate on top.

The transfer from the gels after the electrophoresis to the PVDF membranes was conducted with a constant current of 1.9 mA/cm² for 60 minutes. The PVDF membranes after the completion of the transfer were incubated in a blocking solution (0.5% BSA, 10 mM Tris-HCl, 140 mM NaCl, 0.01% Tween 20, pH 7.5) at room temperature for 60 minutes for blocking operation. After the completion of the blocking, the membranes were incubated and washed twice with a wash buffer (10 mM Tris-HCl, 140 mM NaCl, 0.01% Tween 20, pH 7.5) for five minutes and incubated and then the antibodies of No. 1 to No. 4 were added as primary antibodies which were adjusted at an antibody concentration of 50 μg/ml each, and reacted at room temperature for 90 minutes.

After the completion of the reaction with the primary antibodies, the membranes were incubated and washed twice with the wash buffer for five minutes and incubated and reacted using anti-mouse IgG labeled with an enzyme as the secondary antibody at room temperature for 60 minutes. After the completion of the reaction with the secondary antibody, the membranes were incubated and washed twice with the wash buffer for five minutes and then incubated with a chromogenic substrate at room temperature for 30 minutes, and thus the primary antibodies bound to the dengue virus NS1s transferred to the PVDF membranes were visualized.

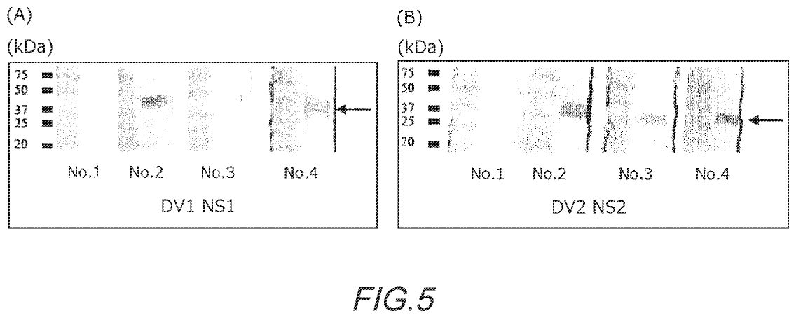

The results are shown in FIG. 5. As shown in FIG. 5, bands of about 42 kDa corresponding to the dengue virus NS were detected with the antibodies of No. 2 to No. 4, but no band was detected when the antibody of No. 1 was used.

### Example 1

Immunochromatography analysis kits each composed of an analyte dilution solution and an immunochromatography analysis device including a sample application part (1), a labeling substance-holding part (2), a chromatography medium part (3) having a detection part (4) and an absorption part (5) were produced.

The antibody of No. 4 produced above was used as the first antibody contained in the labeling substance-holding part, and the antibody of No. 1 produced above was used as the second antibody contained in the detection part. The details are explained below.

(1) Production of Sample Application Part

A nonwoven cloth composed of glass fibers (manufactured by Millipore Corporation: 300 mm×30 mm) was used as the sample application part.

(2) Production of Labeling Substance-Holding Part

To 0.5 ml of a colloidal gold suspension (manufactured by Tanaka Kikinzoku Kogyo K.K.: LC 40 nm), 0.1 ml of the No. 4 antibody which had been diluted to a concentration of 0.05 mg/ml with a phosphate buffer (pH 7.4) was added, and the mixture was left to stand still at room temperature for 10 minutes.

Next, 0.1 ml of a phosphate buffer (pH 7.4) containing 1 mass % BSA was added, and the mixture was further left to stand still at room temperature for 10 minutes. Then, after stirring thoroughly, the mixture was centrifuged at 8000×g for 15 minutes, and the supernatant was removed. Then, 0.1 ml of a phosphate buffer (pH 7.4) containing 1 mass % BSA was added. A labeling substance solution was produced by the above procedures.

A solution obtained by adding 300 μL of a 10 mass % aqueous trehalose solution and 1.8 mL of distilled water to 300 μL of the labeling substance solution produced above was evenly applied to a 12 mm×300 mm glass fiber pad (manufactured by Millipore Corporation) and then dried with a vacuum dryer, and the labeling substance-holding part was thus produced.

(3) Production of Chromatography Medium Part and Detection Part

A sheet composed of nitrocellulose (manufactured by Millipore Corporation, product name: HF120, 300 mm×25 mm) was used as a membrane. Next, 150 μL of a solution obtained by diluting the No. 1 antibody to a concentration of 1.0 mg/ml with a phosphate buffer (pH 7.4) containing 5 mass % isopropyl alcohol was applied to a detection part on the dried membrane in a line with a width of 1 mm using a dispenser for immunochromatography “XYZ3050” (manufactured by BIODOT) at an amount of 1 μL/mm (25 μL per sheet).

Moreover, to check whether the gold nanoparticle labeling reagent has been developed or not and to check the development speed, a solution obtained by diluting goat-derived antiserum having a broad affinity range with the gold nanoparticle labeling substance with a phosphate buffer (pH 7.4) was applied to a control part (a control line) in the downstream of the detection part. Then, by drying at 50° C. for 30 minutes and drying at room temperature overnight, the chromatography medium part and the detection part were produced.

(4) Production of Immunochromatography Analysis Devices

Next, the sample application part, the labeling substance-holding part, the chromatography medium part having the detection part and a nonwoven cloth made of glass fibers as an absorption part for absorbing the developed sample and the labeling substance were attached one by one to a base material composed of a backing sheet. Then, the obtained product was cut with a width of 5 mm with a cutter, and the immunochromatography analysis devices were thus obtained. The length of the labeling substance-holding part in the direction of sample development was adjusted to 12 mm.

(5) Preparation of Analyte Dilution Solution

A 50 mM HEPES buffer (pH 7.5) containing 1 mass % nonionic surfactant (a 1:1 mixture of NP-40 manufactured by Nacalai Tesque, Inc. and Nonidet MN-811 manufactured by NOF Corporation) was prepared and used as the analyte dilution solution for diluting an analyte.

### Example 2

The immunochromatography analysis kits of Example 2 were produced in the same manner as in Example 1 except that the antibody of No. 3 and the antibody of No. 4 were used as the first antibody contained in the labeling substance-holding part and that the antibody of No. 1 and the antibody of No. 2 were used as the second antibody contained in the detection part in Example 1.

### Comparative Example 1

The immunochromatography analysis kits of Comparative Example 1 were produced in the same manner as in Example 1 except that the antibody of No. 1 was used as the first antibody contained in the labeling substance-holding part and that the antibody of No. 4 was used as the second antibody contained in the detection part in Example 1.

The combinations of the antibodies used for the immunochromatography analysis kits of Examples 1 and 2 and Comparative Example 1 are shown in Table 2.

TABLE 2

| | | | Comparative |
|---|---|---|---|
| | Example 1 | Example 2 | Example 1 |
| Labeling | No. 4 Antibody | No. 3 Antibody + | No. 1 Antibody |
| Substance- | | No. 4 Antibody | |
| Holding Part | | | |
| Detection Part | No. 1 Antibody | No. 1 Antibody + | No. 4 Antibody |
| | | No. 2 Antibody | |

### Test Example 1 (Measurement Using Dengue Virus NS1 Recombinant Antigens)

In this test, measurement was conducted using the immunochromatography analysis kits of Examples 1 and 2 produced above and using dengue virus NS1 recombinant antigens as the analytes. DENV-1 to DENV-4 (manufactured by Meridian Life Science, Inc.) were used as the analytes, and analyte-containing solutions were prepared by diluting the analytes to a concentration of 60 ng/mL with the analyte dilution solution.

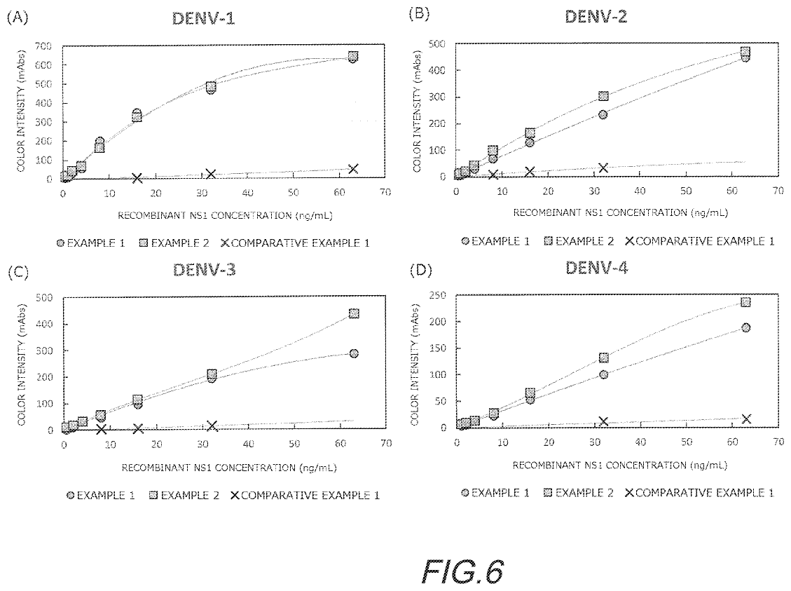

The analyte-containing solutions prepared above in a volume of 90 μL were dropped to the sample application parts of the immunochromatography analysis devices and developed, and the color intensities of the detection parts were measured. SD BIOLINE Dengue NS1 Ag (manufactured by Abbott) was used in Comparative Example 2, and the same test was conducted. The results are shown in FIG. 6.

As shown in FIG. 6, it was found that dengue virus in analytes could be detected with a higher sensitivity with the immunochromatography analysis devices of Examples 1 and 2, in which the labeling substance-holding part contained a first antibody which recognizes the amino acid sequence of SEQ ID NO: 2 that is present in the whole amino acid sequence of dengue virus NS1 of SEQ ID NO: 1 and in which the detection part contained a second antibody which recognizes the three-dimensional structure of dengue virus NS1, as compared to that of Comparative Example 1, in which the labeling substance-holding part contained the second antibody and in which the detection part contained the first antibody. It was also found that the sensitivity to dengue virus of the immunochromatography analysis device of Example 2, in which the first antibody was contained in the detection part, further improved.

### Test Example 2 (Evaluation of Cross-Reactivity)

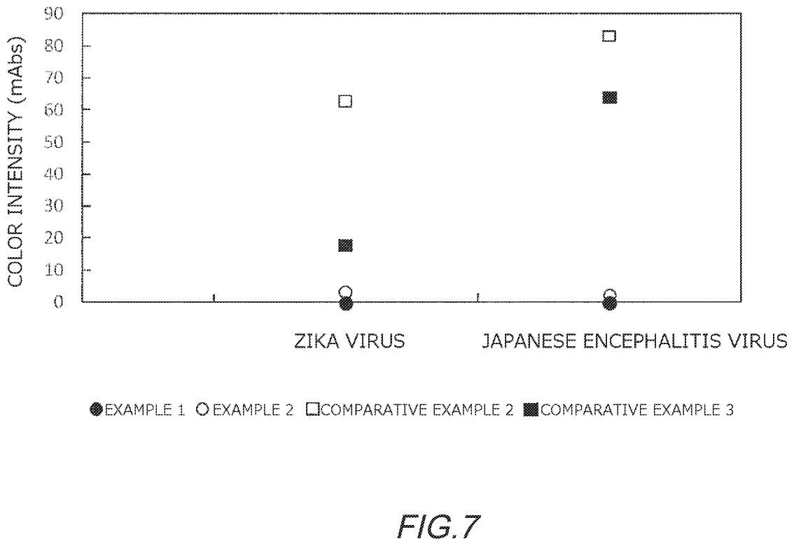

In this test, measurement was conducted using the immunochromatography analysis kits of Examples 1 and 2 produced above and using recombinant Zika virus NS1 (manufactured by Meridian Life Science, Inc., Cat #R01636) or recombinant Japanese encephalitis virus NS1 (Fitzgerald) as the analyte. Analyte-containing solutions were prepared by diluting the analytes with the analyte dilution solution in such a manner that the concentration of the recombinant Zika virus NS1 became 2 mg/mL and that the concentration of the recombinant Japanese encephalitis virus NS1 became 2 mg/mL. The measurement was conducted in the same manner as in Test Example 1 using the prepared analyte-containing solutions as the samples. Moreover, the same test was conducted using SD BIOLINE Dengue NS1 Ag (manufactured by Abbott) in Comparative Example 2 and Panbio (registered trademark) Dengue early rapid (manufactured by Abbott) in Comparative Example 3. The results are shown in FIG. 7.

As shown in FIG. 7, it was found that the immunochromatography analysis kits of the invention did not cause cross-reaction with Zika virus and Japanese encephalitis virus having high homology to dengue virus and could specifically detect dengue virus.

### Test Example 3 (Half-Strip Assay)

By a half-strip assay in which antibody-bound colloidal gold was not applied to a pad and was developed in a nitrocellulose membrane as a liquid, test strips of Example 3 and Comparative Example 4 were produced using the antibodies shown in Table 3 by the procedures shown below, and the S/N ratios were determined.

Using the antibodies shown in Table 3, colloidal gold-labeled antibodies and antibody-immobilized membranes were produced by the same methods as those in Example 1. An antibody-immobilized membrane was attached to a backing sheet, and an absorption pad made of cellulose fibers was attached on the attached antibody-immobilized membrane. Then, the obtained product was cut, and half-strips having a three-layered structure were thus produced.

TABLE 3

| | | Comparative |
|---|---|---|
| | Example 3 | Example 4 |
| Labeling Substance- | No. 4 Antibody | No. 1 Antibody |
| Holding Part | | |
| Detection Part | No. 1 Antibody | No. 4 Antibody |

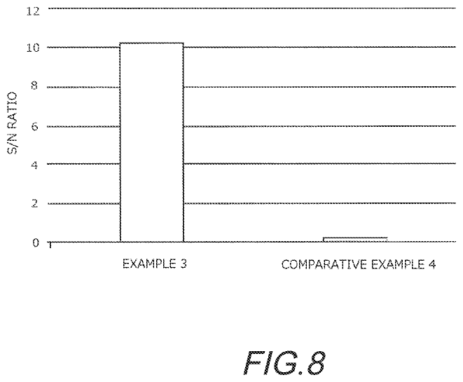

The recombinant NS1 was used as a positive analyte, and PBS was used as a negative analyte. After dispensing 40 μL of the samples to a 96-well plate, Eppendorf tubes or the like, 3 μL of the colloidal gold-labeled antibodies were added. The half-strips were dipped in the mixture solutions of the analyte and the colloidal gold-labeled antibody, and the color intensities of the determination parts were quantified after 15 minutes using an immunochromato reader (manufactured by Hamamatsu Photonics K.K.). The results of the S/N ratios determined from the color values of the negative analyte and the positive analyte are shown in FIG. 8.

As shown in FIG. 8, Example 3, in which the antibody of No. 4 (an antibody which recognizes the amino acid sequence of SEQ ID NO: 2 that is present in the whole amino acid sequence of dengue virus NS1 of SEQ ID NO: 1) was used in the labeling substance-holding part and in which the antibody of No. 1 (an antibody which recognizes the three-dimensional structure of dengue virus NS1) was used in the detection part, showed a significantly higher sensitivity than Comparative Example 4, in which the antibody of No. 1 was used in the labeling substance-holding part and in which the antibody of No. 4 was used in the detection part.

From the results of Reference Examples 1 to 3 and Test Example 3, it is believed that the antibody of No. 1 recognizes the three-dimensional structure of dengue virus NS1. Moreover, it is believed that, when the labeling substance-holding part contains the first antibody, which recognizes the amino acid sequence of SEQ ID NO: 2 that is present in the whole amino acid sequence of dengue virus NS1 of SEQ ID NO: 1, and when the detection part contains the second antibody, the second antibody recognizes and binds through the antigen-antibody reaction to the three-dimensional structure of NS1 to which the first antibody is bound through the antigen-antibody reaction by recognizing the amino acid sequence of SEQ ID NO: 2. It is believed that, because the first antibody first binds to dengue virus NS1, the three-dimensional structure of the site recognized by the second antibody is maintained also during the development and that the state in which the second antibody easily binds to the antigen is maintained, resulting in a significantly high sensitivity.

From the above results, it was found that the immunochromatography analysis device of the invention, in which the labeling substance-holding part contains the first antibody which recognizes the amino acid sequence of SEQ ID NO: 2 that is present in the whole amino acid sequence of dengue virus NS1 of SEQ ID NO: 1 and in which the detection part contains the second antibody which recognizes the three-dimensional structure of dengue virus NS1, can detect dengue virus in an analyte with an excellent sensitivity.

Furthermore, it was found that the immunochromatography analysis device of the invention can specifically detect dengue virus without causing a cross-reaction with another virus having high homology to dengue virus.

Although the invention has been explained in detail referring to specific embodiments, it is obvious to one skilled in the art that various changes and modifications can be made without departing from the spirit and the scope of the invention. The present application is based on a Japanese patent application filed on Jan. 31, 2019 (patent application No. 2019-016494), which is hereby incorporated by reference in its entirety. All the references cited here are incorporated in their entirety.

### REFERENCE SIGNS LIST

1. Sample application part

2. Labeling substance-holding part

3. Chromatography medium part

4. Detection part

5. Absorption part

6. Backing sheet

1. An immunochromatography analysis device for detecting dengue virus in an analyte, including a sample application part, a labeling substance-holding part, a chromatography medium part having a detection part and an absorption part,
wherein the labeling substance-holding part contains a first antibody which recognizes the amino acid sequence of SEQ ID NO: 2 that is present in the whole amino acid sequence of dengue virus NS1 of SEQ ID NO: 1, and
the detection part contains a second antibody which recognizes the three-dimensional structure of dengue virus NS1.

2. The immunochromatography analysis device according to claim 1, wherein the detection part further contains an antibody which recognizes the amino acid sequence of SEQ ID NO: 2 that is present in the whole amino acid sequence of dengue virus NS1 of SEQ ID NO: 1.

3. An immunochromatography analysis kit including the immunochromatography analysis device according to claim 1 and an analyte dilution solution for diluting and developing the analyte.

4. An immunochromatography analysis method for detecting dengue virus in an analyte using the immunochromatography analysis kit according to claim 3, wherein the immunochromatography analysis method includes the following steps (1) to (4),
(1) a step of applying an analyte-containing solution obtained by diluting the analyte with the analyte dilution solution as a sample to the sample application part,
(2) a step of causing the first antibody which recognizes the amino acid sequence of SEQ ID NO: 2 that is present in the whole amino acid sequence of dengue virus NS1 of SEQ ID NO: 1 and which is held in the labeling substance-holding part to recognize dengue virus in the analyte,
(3) a step of developing the analyte and the first antibody as a mobile phase on the chromatography medium part, and
(4) a step of detecting the dengue virus in the developed mobile phase with the second antibody which recognizes the three-dimensional structure of dengue virus NS1 contained in the detection part.


SEQUENCE LISTING (11 sequences)

SEQ ID NO: 1 - protein, 352 aa, Dengue virus
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSA

SEQ ID NO: 2 - protein, 22 aa, Dengue virus
GKKMIRPQPMEHKYSWKSWGKA

SEQ ID NO: 3 - protein, 24 aa, Dengue virus
ELKCGSGIFVTNEVHTWTEQYKFQ

SEQ ID NO: 4 - protein, 17 aa, Dengue virus
IDGPNTPECPDNQRAWN

SEQ ID NO: 5 - protein, 30 aa, Dengue virus
AVHADMGYWIESEKNETWKLARASFIEVKT

SEQ ID NO: 6 - protein, 24 aa, Dengue virus
GGPISQHNYRPGYFTQTAGPWHLG

SEQ ID NO: 7 - protein, 25 aa, Dengue virus
GTTVVVDEHCGNRGPSLRTTTVTGK

SEQ ID NO: 8 - protein, 28 aa, Dengue virus
GEDGCWYGMEIRPVKEKEENLVKSMVSA

SEQ ID NO: 9 - protein, 352 aa, Dengue virus
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTA

SEQ ID NO: 10 - protein, 352 aa, Dengue virus
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSA

SEQ ID NO: 11 - protein, 352 aa, Dengue virus
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTA